(19) United States
(12) Patent Application Publication
(10) Pub. No.: US 2007/0014779 A1
(43) Pub. Date: Jan. 18, 2007

(54) PLASMINOGEN ACTIVATOR VARIANT FORMULATIONS

(75) Inventor: Charles P. Semba, Palo Alto, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/533,305

(22) Filed: Sep. 19, 2006

Related U.S. Application Data
(63) Continuation of application No. 10/697,142, filed on Oct. 30, 2003.
(60) Provisional application No. 60/426,616, filed on Nov. 14, 2002.

(51) Int. Cl. A61K 38/48

(52) U.S. Cl. 424/94.63

(57) ABSTRACT

A solution is provided comprising about 0.01-0.05 mg/mL of tenecteplase in sterile water for injection or bacteriostatic water for injection and normal saline. Such solution is useful for delivery from a catheter and for treating a thrombotic disorder by exposing fibrin-rich fluid from the disorder to an effective amount thereof, as well as in kits. In a preferred embodiment, peripheral thrombosis is treated in a mammal comprising delivering to the mammal via a catheter an effective amount of this solution.

### RELATED APPLICATION

This application is a continuation application filed under 37 CFR 1.53(b)(1), claiming priority under 35 USC 119(e) to non-provisional application number 10\697,142 filed Oct. 30, 2003, which claims priority to provisional application Ser. No. 60/426,616 filed Nov. 14, 2002, the contents of which are incorporated herein by reference.

### BACKGROUND OF THE INVENTION

1. Field of the Invention

This invention relates to solutions of a highly diluted tissue-plasminogen activator variant and methods for treating thrombotic disorders such as peripheral thrombosis using such solutions.

2. Description of Related Disclosures

Many of the common problems in clinical practice today relate to thrombosis. The underlying final pathophysiological process in myocardial infarction and stroke is thrombus formation (thrombogenesis). Common cardiovascular disorders such as atrial fibrillation and heart failure are also associated with thrombogenesis. Thrombosis is also a clinical problem in various cancers and after surgery, as well as in the peripheral and cerebral circulation. As thrombus consists of platelets and fibrin (and often bystanding erythrocytes), optimum anti-thrombotic prophylactic therapy is ideally directed to both.

Peripheral thrombosis is a potentially life-threatening condition caused by a thrombus blocking an artery or vein. Percutaneous or “open” surgical techniques can be used to remove the thrombus. Current percutaneous methods for transluminal removal of thrombus (TRT) include thrombolytic therapy (i.e., catheter-directed, pharmacomechanic), percutaneous aspiration thrombectomy (PAT), and percutaneous mechanical thrombectomy (PMT). These methods may be used in combination.

Catheter-directed thrombolytic therapy has at least three theoretical and practical advantages over surgical thromboembolectomy: less endothelial trauma, angiographic visualization of the underlying lesion(s) and runoff vessels, and, in many cases, ready access for definitive transluminal therapies that address the underlying lesion. In addition, it has been suggested that gradual, low-pressure reperfusion may offer certain advantages over sudden, high-pressure reperfusion associated with surgical revascularization.

Since catheter-directed thrombolysis is the preferred treatment (see Results of a prospective randomized trial evaluating surgery versus thrombolysis for ischemia of the lower extremity: STILE trial. Ann. Surg., 220: 251-268 (1994)), prior to 1999, human-derived urokinase was virtually the sole agent used for peripheral thrombolysis. With the unavailability of urokinase, beginning in early 1999, the peripheral vascular interventional community began to look for alternative agents for thrombolysis.

Plasminogen activators are enzymes that cleave the peptide bond of plasminogen between amino acid residues 561 and 562, converting it to plasmin. Plasmin is an active serine proteinase that degrades various proteins, including fibrin.

Currently, five plasminogen activators are approved in the United States for treating coronary thromboses, but none are FDA-approved for catheter-directed thrombolysis. In the past three years, significant clinical research has been performed with use of recombinantly derived agents for catheter-directed therapy (CDT). Techniques have been refined and treatment of deep vein thrombosis (DVT) has been reported to be effective and safe with all available plasminogen activators in non-randomized, non-controlled observational studies (Elsharawy and Elzayat, Eur. J. Vasc. Endovasc. Surg., 24: 209-214 (2002); Semba and Dake, Radiology, 191: 487-494 (1994); Chang et al., J. Vasc. Interv. Radiol., 12: 247-252 (2001); Castaneda et al., J. Vasc. Interv. Radiol., 13: 577-580 (2002); Semba et al., Tech. Vasc. Interv. Radiol., 4: 99-106 (2001); Allie et al., Am. J. Cardiol., 90 (suppl 6A): 108H (2002)).

An early review of the literature suggested that the major complication rate undergoing thrombolysis with recombinant tPA for peripheral arterial occlusive disease was 5.1% (Semba et al., J. Vasc. Interv. Radiol., 11: 149-161 (2000); Swischuk et al., J. Vasc. Interv. Radiol., 12: 423-430 (2001)). A recent tPA trial at a dose of 0.04 mg/kg/hr found major complications of 13% (Arepally et al., J. Vasc. Interv. Radiol., 13: 45-50 (2002)).

Initial results of reteplase in the treatment of acute lower extremity arterial occlusions showed a mortality rate of 6% with a currently employed low-dose regimen of 0.5 u/hour (Davidian et al., J. Vasc. Interv. Radiol., 11: 289-294 (2000)). More recently, a pilot study of reteplase employed for thrombolysis of deep venous thrombosis reported a major complication rate of 4% (Castaneda et al., supra).

Tenecteplase (TNK, TNKASE™, Genentech, Inc., South San Francisco, Calif.) is available in a commercially supplied 50-mg vial and approved for a single-bolus administration in patients with acute myocardial infarction (AMI) (TNKASE™. Full prescribing information. 2002 Physicians Desk References Thomas Medical Economics Co., Montvale, N.J.). Tenecteplase is a genetically engineered variant of human tPA cloned and expressed in CHO cells (Keyt et al., Proc. Natl. Acad. Sci USA., 91, 3670-3674 (1994)). See also Verstraete, Am. J. Med., 109: 52-58 (2000) for an overview of third-generation thrombolytic drugs in general. When used in approved indications, tenecteplase is reconstituted in sterile water to achieve a final concentration of 5 mg/mL and administered intravenously as a single weight-adjusted bolus.

In pre-clinical studies, tenecteplase has demonstrated increased potency, higher fibrin specificity, resistance to plasminogen activator inhibitor (PAI-1), and faster clot lysis, with less systemic fibrinolysis, plasminogenemia, and bleeding compared to alteplase (Refino et al., Thromb. Haemost., 70: 313-319 (1993); Keyt et al., supra; Collen et al., Thromb. Haemost., 72: 98-104 (1994); Patel et al., J. Vasc. Interv. Radiol., 12: 559-570 (2001)); Benedict et al., Circulation, 92: 3032-3040. (1995)).

In human clinical trials for treatment of acute myocardial infarction, tenecteplase demonstrated similar efficacy to alteplase, but major blood loss was reduced by 22%, need for blood transfusion was reduced by 23%, and minor bleeding decreased by 16% (Assessment of the Safety and Efficacy of a New Thrombolytic Investigators (ASSENT-2). Single-bolus tenecteplase compared with front-loaded alteplase in acute myocardial infarction: the ASSENT-2 double blind randomized trial. Lancet, 354: 716-722 (1999)). There was no significant difference in the rate of intracranial hemorrhage (0.9%). Because of the superior safety profile seen in AMI and its increased clot lysis efficiency, investigators have been exploring the off-label use of tenecteplase in non-coronary applications as an alternative thrombolytic agent (Semba et al., Tech. Vasc. Interv. Radiol., (2001), supra; Sze et al., J. Vasc. Interv. Radiol., 12: 1456-1457 (2001); Razavi et al., J. Vasc. Interv. Radiol., 13: (2), Part 2: S11 (February 2002); Nehme et al., J. Vasc. Interv. Radiol., 13: S109 (2002)).

Prior to 1999 few interventionalists had significant experience with recombinant agents, and little was understood of their storage and handling, titration range, and dosing limits. Initial reports using alteplase were complicated by adverse bleeding events due to infusion protocols involving high doses (as high as 0.1 mg/kg/hr) of undiluted alteplase in an attempt to deliver an appropriate amount of effluent through the infusion catheter (Ouriel et al., J. Vasc. Interv. Radiol., 11: 295-298 (2000); McNamara et al., Am. J. Cardiol., 84: 37P (1999)). With increased experience, investigators began using diluted solutions of alteplase to maintain high volumes of effluent but lower hourly delivered doses of the agent (Valji, J. Vasc. Interv. Radiol., 11: 411-420 (2000); Semba et al., Tech. Vasc. Interv. Radiol., (20.01), supra; Semba et al., J. Vasc. Interv. Radiol., 11: 279-287 (2000)). Some investigators have reported clinical efficacy and minimal adverse events using targeted alteplase dilutions up to 0.002 mg/mL (Semba et al., supra, J. Vasc. Interv. Radiol., (2000) pp. 279-287).

In attempts to determine the optimal dilution of alteplase, Bookstein demonstrated that a 0.01 mg/mL concentration provided maximal clot-lysis using a pulse-spray rabbit model and the degree of lysis was further enhanced by addition of exogenous plasminogen (Bookstein and Bookstein, J. Vasc. Interv. Radiol., 11: 299-303 (2000); Bookstein and Bookstein, J. Vasc. Interv. Radiol., 11: 1353-1362 (2000)).

For catheter-directed therapy, lyophilized tenecteplase is reconstituted in sterile water (5 mg/mL) and further diluted in normal saline (0.01 to 0.25 mg/mL) (Semba et al., Tech. Vasc. Interv. Radiol., supra, (2001); Allie et al., supra); Razavi et al., supra; Semba et al., J. Vasc. Interv. Radiol., 13: (2), Part 2: S75 (February 2002). Specifically, Razavi et al., supra, reports that using a 0.01 mg/mL dilution of tenecteplase in normal saline infused at 25 to 50 mL/hr (0.25-0.5 mg/hr) results in angiographic efficacy in arterial and venous clot lysis (Razavi et al., supra). Additionally, Razavi et al., J. Endovasc. Ther., 9:593-598 (2002) disclose that such doses of tenecteplase are safe and effective in peripheral catheter-directed thrombolytic therapy of arterial occlusions and deep vein thrombosis. Semba et al., J. Vasc. Interv. Radiol., (2002), page S75, supra, discloses that these solutions are stable and bioactive and confirms that tenecteplase can be frozen and stored in small aliquots. Tenecteplase is the only agent to show a superior safety profile to tPA with a significant reduction in non-intracranial major bleeding and the need for blood transfusions (Benedict et al., supra; ASSENT-2, Lancet, supra). In addition, Allie et al., Tenecteplase in Peripheral Thrombolysis: Initial Safety and Feasibility Experience, abstract 48 of Society of Interventional Radiology, March 2003 (page S17) discloses that continuous TNK infusion (0.25 to 0.50 mg/hour) is a safe and feasible treatment for peripheral chemical thrombolysis. Further, tenecteplase diluted to a 0.0125 mg/ml solution was found to be a feasible treatment for thrombolysing occluded peripheral arteries and veins, with only moderate effect on fibrinogen levels (Burkart et al., J. Vasc. Interv. Radiol., 13: 1099-1102 (2002)), and when combined with eptifibatide, was found to be a feasible treatment for thrombolysing acute peripheral arterial and venous occlusions (Burkart et al., J. Vasc. Interv. Radiol., 14: 729-733 (2003)). Hence, tenecteplase has been tested at various concentrations in peripheral applications.

Regarding ex vivo or in vitro catheter clearance, the current standard care of vascular catheters includes flushing the lumen of the catheter with an anti-coagulant, such as heparin, to prevent blood in and around the tip of the catheter from coagulating and obstructing the flow of fluids through the catheter. Furthermore, heparin has no anti-microbial activity, and, in addition, if not carefully controlled, it can carry the anti-coagulation process too far, thereby presenting a risk of hemorrhage. Heparin can also result in antibody formation, leading to a serious autoimmune condition of heparin-induced thrombocytopenia (HIT), which depletes platelets and further increases risk of bleeding. Thus, infections, as well as thrombotic occlusion, continue to occur frequently despite the prophylactic use of heparin flushes. Knowledge of the pathogenesis and microbiology of central venous catheter-related infections is essential to provide effective prevention of this problem. CATHFLO™ tPA has been developed to aid in catheter clearance, and has proven effective for this purpose.

Notwithstanding the above-described contributions to the art, the current tenecteplase formulation being sold may not be conducive to any indications involving thrombotic therapy, including catheter clearance and catheter-directed therapy, because of its high concentration and significantly higher potency compared to tPA. There exists a need to devise a tenecteplase solution useful in thrombotic disorders. For example, there is a need for such solution for catheter-directed thrombolysis in a clinical setting that allows lower overall doses, but very high concentrations delivered to the clot through a catheter, which may be embedded in the clot. Such an invention would increase the efficacy of surface exposure of the clot to the tenecteplase.

Furthermore, a need continues to exist for the following method of treating a thrombotic disorder: a non-toxic method for removal of fibrin-bound blood clots from indwelling medical devices. There is a further need for the prevention and removal of fibrin from such devices, as certain bacteria have binding sites that favor sticking to fibrin, in particular.

### SUMMARY OF THE INVENTION

Accordingly, the invention is as claimed. A dosing and dilution scheme for tenecteplase for use in treating pathological collections of fibrin-rich fluid such as thrombolysis and catheter cleansing is devised. Reconstituted frozen/thawed and highly diluted tenecteplase solutions are found to be stable in vitro and fully bioactive.

In one embodiment of the invention herein, a solution is provided comprising about 0.01 to 0.05 mg/mL of tenecteplase in sterile water for injection or bacteriostatic water for injection and normal saline. In a further embodiment, a catheter is provided comprising the solution.

In another embodiment, the invention provides a method for treating a pathological collection of a fibrin-rich fluid comprising exposing the fluid to an effective amount of a solution comprising about 0.01 to 0.05 mg/mL of tenecteplase in sterile water for injection or bacteriostatic water for injection and normal saline.

In a specific embodiment of this method, the invention provides a method for treating peripheral thrombosis in a mammal comprising delivering to the mammal via a catheter an effective amount of a solution comprising about 0.01 to 0.05 mg/mL of tenecteplase in sterile water for injection or bacteriostatic water for injection and normal saline.

In a specific preferred aspect, the above method further comprises administering to the mammal an effective amount of another agent for treating the thrombosis.

In another aspect, a kit is provided comprising a container comprising lyophilized tenecteplase, a container comprising sterile water for injection or bacteriostatic water for injection, a container comprising normal saline, and instructions for reconstituting the tenecteplase with the water for injection and diluting the reconstituted tenecteplase with the normal saline to a final concentration of about 0.01 to 0.05 mg/mL of tenecteplase. In one preferred embodiment, the container with tenecteplase contains about 10-50 mg tenecteplase, the container with water for injection contains about 2-10 mL of such water, and the instructions indicate that the tenecteplase is reconstituted to a final concentration of about 5 mg/mL, and more preferably about 10 mg tenecteplase for 2 mL water or about 50 mg tenecteplase for 10 mL water.

In an alternative kit embodiment, the kit comprises a container comprising a solution comprising about 0.01 to 0.05 mg/mL of tenecteplase in sterile water for injection or bacteriostatic water for injection and normal saline, and instructions for exposing the solution in an effective amount to a pathological collection of a fibrin-rich fluid.

The invention herein hence provides a highly diluted tenecteplase solution useful in treating pathological collections of fibrin-rich fluids such as catheter-related disorders, for example, catheter-directed thrombolysis. This can be in a clinical setting that allows lower overall doses, but very high concentrations delivered to the clot through a catheter, which may be embedded in the clot. Such an invention increases the efficacy of surface exposure of the clot to the tenecteplase.

Furthermore, highly dilute solutions offer a safer method to deliver tenecteplase when used in treating thrombotic disorders, since the dose is lower. For CDT specifically, the dilution allows the operator to deliver a high volume of effluent through the catheter but at a more controlled dose.

### DESCRIPTION OF THE PREFERRED EMBODIMENTS

Definitions

As used herein, a “pathological collection of a fibrin-rich fluid,” or “thrombotic disorder” refers to any disorder involving the collection of a fluid from any source, including blood, cerebrospinal fluid, urine, and fluid from the peritoneal, pleural, or pericardial cavity, that can be treated with a thrombolytic drug. Hence, this disorder includes intra-vascular as well as non-vascular collections of fluid. This collection of fluid may be contained in any vehicle capable of holding and/or circulating such fluid, such as, for example, an artery, vein, stent, or catheter. Thus, the collection may be in vitro, ex vivo, or in vivo, and the thrombus may be present in any artery or vein, whether in the coronary, cerebral, or peripheral circulation.

This term includes disorders in mammals that can be treated by thrombolytic therapy, such as catheter-directed, intravenous, and inhalation therapy, and catheters that are occluded due to a thrombus or other reasons. Examples of such disorders include, without limitation, peripheral thrombosis, occluded central venous catheters and vascular ports, indwelling shunts (e.g., ventriculoperitoneal shunts), thrombosed arterial cannulas, pulmonary catheters, hemodialysis access, intra-arterial management of acute ischemic stroke, intra-arterial management of any end organ that can be cannulated with a catheter (e.g., acute embolic ischemia of the kidney or mesenteric circulation), intra-catheter instillation for treatment of loculated pleural effusion, hemothorax, empyema, and lung abscess, catheter-directed treatment of infected fluid collections (e.g., abscess cavities and loculated infections), fibrin depositions involved in sepsis or acute respiratory distress syndrome (ARDS), since the pathophysiology of ARDS (and sepsis) involves microthrombi developing in the small vessels of the lungs, kidneys, and other organs (see Gram et al., Fibrinolysis and Proteolysis, 13: 209-212 (1999) and Hardaway et al., American Surgeon, 67: 377-382 (2001)), and intraventricular hemorrhage of the brain. The preferred disorder for treatment herein is peripheral thrombosis.

As used herein, “peripheral thrombosis” refers to a blood clot in any part of the body except a coronary clot. It is preferably an arterial or venous thrombosis. It could be, for example, a clot formed in a leg artery or an arm vein. When thrombosis is treated, it is called thrombolysis, since the clot is lysed. Thrombosis includes limb ischemia, which is any sudden decrease or worsening in limb perfusion that causes a potential threat to limb viability. In situ thrombosis or embolus may cause acute peripheral arterial occlusion. Catheter-directed thrombolysis herein includes lysis in deep venous thrombosis, iliofemoral venous or deep venous thrombosis, venous thrombosis of the lower extremity, thrombolytic therapy in limb salvage, treatment of lower extremity arterial and graft occlusions, and treatment of acute vascular occlusion.

As used herein, “treating” describes exposure of the pathological collection to the solution herein, whether in vitro, ex vivo, or in vivo. In the in vitro or ex vivo context, it describes the exposure of the thrombus-containing fluid to the solution in such a way as to lyse the thrombus. Preferably, the fluid is combined in a catheter, which is preferably flushed with the solution.

In the in vivo context, it describes the management and care of a mammal, especially a human patient, for the purpose of combating a thrombotic disorder and includes the administration to prevent the onset of the symptoms or complications of, alleviate the symptoms or complications of, or eliminate the thrombotic disorder. For ARDS or sepsis, preferably the treatment is by inhalation or intravenous. For peripheral thrombosis, the pathological collection is preferably contained within a catheter, which is more preferably indwelling.

For purposes of this invention, beneficial or desired clinical results for treating peripheral thrombosis include, but are not limited to, increased percent clot lysis, determined, for example, by angiographically documented reduction in clot burden after the termination of tenecteplase infusion. Percent clot lysis is estimated based on the difference between the volume of the thrombus before and after lysis.

Additionally, technical success for arterial peripheral thrombosis may be defined as restoration of antegrade flow and >95% removal of thrombus on follow-up arteriogram (Patel et al., supra), and clinical success as immediate limb salvage and relief of ischemic rest pain. Clinical failure in arterial cases can be defined as the need for surgical intervention in the affected extremity because of failure of clot lysis, or occlusion of outflow runoff vessels. Technical success for venous peripheral thrombosis can be defined as restoration of antegrade flow and >95% removal of thrombus on follow-up venogram (Patel et al., supra), and clinical success as resolution or improvement in extremity pain and swelling, while clinical failure can be defined as unchanged or worsening of extremity pain and swelling.

As will be understood by one of skill in the art, the particular symptoms or conditions that yield to treatment in accordance with the invention will depend on the type of disorder being treated. Those “in need of treatment” include mammals already having the disorder, as well as those prone to having the disorder, including those in which the disorder is to be prevented.

The term “mammal” for the purposes of treatment refers to any animal classified as a mammal, including but not limited to, humans, sport, zoo, pet, and domestic or farm animals, such as dogs, cats, cattle, sheep, pigs, horses, and primates, such as monkeys or humans. Preferably the mammal is a human.

A “therapeutic composition” or “composition,” as used herein, is defined as comprising tenecteplase, sterile water for injection or bacteriostatic water for injection, and normal saline, as well as any optional pharmaceutically acceptable carrier(s), such as minerals, proteins, and other excipients known to one skilled in the art.

As used herein, “catheter” refers to a medical device generally, but not necessarily, constructed of plastic polymers, e.g., polyurethane, silicone, or other polymers, that is useful in catheter-directed therapy (i.e., delivering medical therapy) to treat peripheral thrombosis. Alternatively, it is used to treat any pathological collection of a fibrin-rich fluid.

Such catheters represent a wide variety of medical devices that may be external or indwelling, such as, for example, a urinary catheter, a vascular catheter, an arterial catheter, a peritoneal catheter, a tracheal catheter, a Swan-Ganz catheter, a hemodialysis catheter, an umbilical catheter, a ventriculostomy or intrathercal catheter, a percutaneous non-tunneled silicone catheter, a cuffed tunneled central venous catheter, as well as a subcutaneous central venous port, and the like. Useful catheters include the catheter with a balloon inflation lumen as described in U.S. Pat. No. 5,865,178, and in U.S. application 2001/0021811 published Sep. 13, 2001 for introducing clot-dissolving drugs, provided they can be used in thrombolytic applications.

“Vascular catheter” is used herein to describe a catheter involving the vascular system and includes peripheral catheters and central venous catheters (CVCs), long-term cuffed devices, short-term, non-cuffed devices, and implantable ports. CVCs, which are the same as central venous access devices (CVADs), include peripherally inserted central catheters (PICC lines), external cuffed devices, non-cuffed catheters, ventriculostomy or intrathercal catheters, non-tunneled and subcutaneously tunneled catheters, hemodialysis (HD) catheters, and ports.

The preferred catheters herein are an indwelling catheter, such as an intravenous or arterial catheter or a CVC, preferably a cuffed tunneled CVC, a peripheral intravenous catheter, an arterial catheter, a Swan-Ganz catheter, a hemodialysis catheter, an umbilical catheter, a percutaneous non-tunneled silicone catheter, a ventriculostomy or intrathercal catheter, or a subcutaneous central venous port. Also preferred is that the catheter be inserted directly into the thrombus being treated. Specific preferred examples of catheters include a 6-F, 100-cm ANGIOJET™ XPEEDIOR™ catheter (Possis, Minneapolis, Minn.) and a UNIFUSE™ catheter (AngioDynamics, Queensbury, N.Y.).

As used herein, “solution” refers to a soluble mixture of ingredients, including complete solvation of the ingredients.

As used herein, “preparation” refers to a composition, solution, formulation, or the like that is an admixture of the ingredients set forth.

As used herein, the term “tenecteplase,” also known as TNK-tPA or TNKASE™ brand of tissue-plasminogen activator variant, refers to a tPA variant designated T103N, N117Q, K296A, H297A, R298A, R299A available from Genentech, Inc. wherein Thr103 of wild-type tPA is changed to Asn (T103N), Asn117 of wild-type tPA is changed to Gln (N117Q), and Lys-His-Arg-Arg (SEQ ID NO:1) 296-299 of wild-type tPA is changed to Ala-Ala-Ala-Ala (SEQ ID NO:2)(KHRR296-299AAAA). See the background section herein and U.S. Pat. No. 5,612,029.

As used herein, “co-agent for treating the pathological collection” refers to an active drug or material other than tenecteplase that can be used to treat the pathological collection herein. Examples include drotrecogin alfa (activated), a recombinant form of human Activated Protein C made by Eli Lilly & Co. sold under the name XIGRIS™, synthetic cerebrospinal fluid (e.g., NEURYSOL™—Neuron Therapeutics, Inc.), and endovascular coiling of aneurysms, as well as the co-agents for treating the thrombosis as defined herein.

As used herein, “co-agent for treating the thrombosis” refers to an active drug or material other than tenecteplase that can be used to treat peripheral thrombosis. Examples of such agents include blood thinners such as heparin and heparin analogs including low-molecular-weight heparin such as tinzaparin, certoparin, parnaparin, nadroparin, ardeparin, enoxaparin (LOVENOX™, Aventis Pharma, Bridgewater, N.J.), reviparin, reviparin and dalteparin, warfarin (3-(alpha-acetonylbenzyl)-4-hydroxycoumarin, or COUMADIN®), or aspirin; anti-coagulants such as tPA; tPA variants such as reteplase; urokinase; streptokinase; alfimeprase; glycoprotein (GP) IIb/IIIa platelet receptor antagonists; and other antiplatelet drugs.

There are currently three FDA-approved intravenous GPIIb-IIIa inhibitors: abciximab (REOPRO™, Centocor, Malvern, Pa.), eptifibatide (INTEGRILIN™, COR Therapeutics, South San Francisco, Calif.), and tirofiban (AGGRASTAT™ Merck, White House Station, N.J.). These drugs prevent platelet aggregation by binding the GPIIb-IIIa receptor. Further information concerning such inhibitors, including dosing and combination therapy, can be found, for example, in Hofmann et al., Cardiovasc. Interv. Radiol., 24: 361-367 (2001). Specific examples of useful GPIIb/IIIa antagonist compounds are roxifiban, abciximab, eptifibatide, tirofiban, lamifiban, lefradafiban, sibrafiban (Ro-48-3657), orbofiban and xemilofiban (see, for example, Graul et al., Xemilifiban; Drugs of the Future, 22: 508-517 (1997); Scarborough, Eptifibatide. Drugs of the Future, 23: 585-590 (1998)), as well as certain such inhibitors having chemical formulae as described in U.S. Pat. No. 6,346,517 and in WO 00/53264, as well as in WO 95/14683, and U.S. Pat. No. 5,849,736. Of these, abciximab, eptifibatide, tirofiban are preferred. Others will be readily apparent to those skilled in the art.

Other such agents include direct thrombin inhibitors, such as argatroban, lepirudin, or bivalirudin, as well as clopidogrel and ticlopidine. In addition, such agents include Factor Xa inhibitors, including both peptide and non-peptide Factor Xa inhibitors (Kaiser, Thrombin and factor Xa inhibitors. Drugs of the Future, 23: 423-436 (1998)). Examples of such peptide inhibitors include antistasin and tick anticoagulant peptide, and examples of such non-peptide inhibitors are described, for example, in WO 98/02326 and in Thromb. Haemost., 71: 314-319(1994); Thromb. Haemost., 72: 393-396 (1994); and Thromb. Haemost., 79: 859-864 (1998). As used herein, “sterile water for injection” or “SWFI” refers to the same substance as defined by the United States Pharmacopeia (USP), which is a sterile, nonpyrogenic preparation of water for injection that contains no bacteriostat, antimicrobial agent, or added buffer and is supplied only in single-dose containers to dilute or dissolve drugs for injection.

“Normal saline” refers to an aqueous solution of water containing 0.9% sodium chloride. It is also known as 0.9% sodium chloride injection USP, non-heparinized normal saline. Such saline is generally used clinically as a diluent for drugs administered by injection and as a plasma substitute.

“Bacteriostatic water for injection” or “BWFI” refers to a mixture of water and varying amounts of benzyl alcohol with no other ingredients as defined by the United States Pharmacopeia (USP).

### MODES FOR CARRYING OUT THE INVENTION

A stable formulation of tenecteplase is presented in the form of a solution comprising tenecteplase, sterile water for injection or bacteriostatic water for injection, and normal saline, wherein the tenecteplase is present in a concentration of about 0.01 to 0.05 mg/ml. This concentration range represents an effective dosage for treating thrombotic disorders, including for catheter delivery. Preferably, the amount of tenecteplase is about 0.01 to 0.04 mg/mL, more preferably about 0.01 to 0.03 mg/mL, still more preferably about 0.01 to 0.02 mg/mL, even more preferably about 0.01 to 0.015 mg/mL, even still more preferably about 0.01 to 0.014 mg/mL, still more preferably about 0.01 to 0.013 mg/mL, still more preferably about 0.01 to 0.012 mg/mL, still more preferably about 0.01 to 0.011 mg/mL, and most preferably about 0.01 mg/mL.

The amount of tenecteplase provided is that which will effect thrombolysis in a clinical or medical setting (with clinical and technical endpoints such as those set forth above in the definition section), but will not exceed that which would be a dangerous level in vivo so as to cause bleeding or other complications. Examples of major adverse events include intracranial hemorrhage; bleeding at any site requiring evacuation, transfusion, or prolonging hospital stay; any procedure-related adverse event requiring additional procedures; or death as a complication of the thrombolytic procedure. Examples of minor bleeding complications include an access site hematoma greater than 5 cm that did not require any specific treatments or bleeding at any site and/or that was managed conservatively without the need for transfusion, evacuation, or prolongation of hospital stay.

Compositions particularly well suited for the clinical administration of the tenecteplase used to practice this invention include sterile aqueous solutions or sterile hydratable powders such as lyophilized protein. Typically, an appropriate amount of a pharmaceutically acceptable salt is also used in the formulation to render the formulation isotonic. A buffer such as arginine base in combination with phosphoric acid is also typically included at an appropriate concentration to maintain a suitable pH, generally from 5.5 to 7.5. In addition or alternatively, a compound such as glycerol may be included in the formulation to help maintain the shelf-life. More preferably, this formulation, which comprises from about 0.01 to about 0.05 mg/mL tenecteplase, sterile water for injection or bacteriostatic water for injection, and normal saline, preferably also comprises arginine, phosphoric acid, and an emulsifying agent such as a polyoxyethylene sorbitan fatty ester such as POLYSORBATE 20™ polyoxyethylene 20 sorbitan monolaurate, POLYSORBATE 80™ polyethylene sorbitan monooleate, or POLYSORBATE 65™ polyoxyethylene 20 sorbitan tristearate, which accompany the tenecteplase that is lyophilized.

As one example of an appropriate dosage form, a vial containing about 50 mg of tenecteplase, as well as arginine, phosphoric acid, and a POLYSORBATE™ emulsifier is reconstituted with 50 ml of sterile water for injection and mixed with a suitable volume of 0.9 percent sodium chloride injection.

Tenecteplase is commercially available as a sterile, preservative-free, lyophilized powder in a vial containing 52.5 mg of tenecteplase, 0.55 g of L-arginine, 0.17 g of phosphoric acid, 4.3 mg of POLYSORBATE 20™ polyoxyethylene 20 sorbitan monolaurate supplied with a 10-mL syringe of sterile water for injection USP. Alternatively, the tenecteplase can be supplied with a 10-mL syringe of bacteriostatic water for injection. The preferred water for injection herein is sterile water for injection.

To reconstitute the product, 10 mL of preservative-free sterile water for injection USP is mixed with the tenecteplase powder under sterile conditions to produce a final concentration of 5 mg/mL. Alternatively, tenecteplase is reconstituted to 5 mg/mL in BWFI (0.9%) with full viability of protein. Unused reconstituted tenecteplase may be stored at controlled room temperature (15-30° C.) for up to 8 hours or under refrigeration (2-8° C.) for 24 hours.

Alternatively, in a small vial configuration, a vial may contain 10 mg of tenecteplase in a 10-mg vial with a 2-mL vial of the water for injection. Vials with weights and volumes in between about 10 mg and 50 mg and between about 2 mL and 10 mL, respectively, are contemplated herein.

Thrombolytic procedures using tenecteplase typically involve total doses significantly less than about 50 mg (e.g., 2 mg for catheter clearance), and reconstituted tenecteplase may be frozen for later use. Many institutions are reconstituting and freezing smaller aliquots (2- and 5-mL syringes) of tenecteplase for future use to minimize waste and decrease costs.

According to the invention herein, reconstituted tenecteplase is diluted in normal saline to about 0.01 to 0.05 mg/mL, preferably only in normal saline and not other ingredients. Tenecteplase may be diluted to a concentration of about 0.01 mg/mL without triggering precipitation and affecting biochemical stability at 25° C. for 8 hours.

Visual inspection of the solution for precipitates is recommended after dilution and before administration. To minimize the risk of precipitate formation from excessive dilution in cases in which large volumes of normal saline or higher rates of infusion are necessary, the tenecteplase solution is preferably piggybacked into a normal saline infusion just before entering the infusion catheter.

Tenecteplase is preservative-free and is theoretically susceptible to bacterial contamination and biochemical degradation when left at room temperature for more than 8 hours. Although the manufacturer recommends changing the solution after 24 hours, the drug should be physically and chemically stable for 24 hours.

If fibrin-bound blood clots are being removed from a catheter the catheter may be contacted with the solution herein for at least about 5 days, preferably about 6 to 15 days.

The tenecteplase solution herein may be contacted with the pathological collection by any suitable technique. If treatment is in vivo, the solution is administered to the mammal via, e.g., oral, parenteral (e.g., intramuscular, intraperitoneal, intravenous, intra-articular, or subcutaneous injection or infusion, or implant), nasal, pulmonary, vaginal, rectal, or sublingual routes of administration, and can be formulated in dosage forms appropriate for each route of administration. The specific route of administration will depend, e.g., on the medical history of the patient, including any perceived or anticipated side effects using the solution and the particular type of disorder to be corrected.

In one specific embodiment, the tenecteplase solution may be administered parenterally to patients suffering from thrombolytic diseases or conditions, or by other methods that ensure its delivery to the bloodstream in an effective form. Most preferably, the administration is by catheter, inhalation/nebulizer, continuous infusion (using, e.g., slow-release devices or minipumps such as osmotic pumps or skin patches), or injection (using, e.g., intravenous, intra-articular or subcutaneous means). Depending on the particular indication, the solution may be administered locally, for example, directly to the location where lysis is needed.

Specifically, if the thrombotic disorder is peripheral thrombosis, the tenecteplase solution is generally delivered through a catheter, preferably an indwelling catheter, and preferably directly to the clot, i.e., placed in the blood clot. If the indication is sepsis or ARDS, the tenecteplase solution may be aerosolized or nebulized and inhaled for treating fibrin depositions that may occur, or it may be administered intravenously. If the indication is brain- or spinal-related, the tenecteplase solution may be instilled through a catheter placed neurosurgically into the caverns of the brain or spinal cord, etc. The skilled practitioner would be easily able to devise methods of administration of the solution herein based on the general knowledge in the extensive literature on uses of alteplase, tenecteplase, and other lytics.

The solution to be used in in vivo therapy will be dosed in a fashion consistent with good medical practice, taking into account the clinical condition and age of the individual mammal, preferably patient (especially the side effects of treatment with the solution), the type of disorder, the site of delivery, the chosen route of administration, the scheduling of administration, and other factors known to practitioners. The effective amounts of the tenecteplase solution for purposes herein are thus determined by such considerations and must be amounts that result in bioavailability of the drug to the mammal and the desired effect.

A “therapeutically effective” amount for purposes of treating peripheral thrombosis herein is determined by the above factors, but is generally about 0.001 to 0.2 mg/kg body weight/hour. The preferred dose is about 0.001-0.1 mg/kg/hour, more preferably about 0.001 to 0.05 mg/kg/hour.

The effects of administration of tenecteplase can be measured by a variety of assays known in the art, as noted in the definitions above, such as the percent clot lysis assay.

For catheter-directed thrombolysis for acute limb ischemia (greater than 14 days), the following dosage regimens serve as current guidelines:

(i) weight-based dose: about 0.003 to 0.01 mg/kg/h, and

(ii) non-weight-based dose: about 0.25 to 0.5 mg/h.

The total hourly-infused dose preferably does not exceed about 1 mg/h with the use of either method.

Though the maximum manufacturer-recommended tenecteplase dose is 50 mg for treating acute myocardial infarction, the recommended total dose is predicted to be about 20 mg for catheter-directed therapy. If a single-bolus dose is preferred, the dose preferably does not exceed about 5 mg. Much lower doses may be equally effective, although time to lysis may be longer.

For treating ischemia, tenecteplase is expected to be equally efficacious whether administered with use of continuous end-hole delivery, intrathrombic infusion through a multi-side-hole catheter, bolus techniques, or pulse-spray thrombolysis. Therefore, physicians should continue to use the catheter-based modality with which they are most comfortable.

The most common complication of thrombolytic therapy is adverse bleeding. Monitoring of fibrinogen levels is commonly used during prolonged lytic infusions, and the thrombolytic agent is often titrated (usually downward) to maintain the fibrinogen level above 100 mg/dL. Despite the frequency of this practice, there is no pivotal study to support that fibrinogen levels are predictive of adverse bleeding; hemorrhagic complications can occur with values above 100 mg/dL. In the future, a more appropriate parameter may be the 2-antiplasmin levels; however, this remains to be validated in clinical trials.

Risk factors for adverse bleeding with tenecteplase are similar to those associated with alteplase, UK, and other plasminogen activators. Variables associated with adverse bleeding risks include increased tenecteplase dose, duration of infusion, adjunctive anti-thrombotic therapy (e.g., heparin, aspirin, or other anti-platelet agents as noted herein), hypertension, increasing age, severity of ischemia, and female gender. Physicians should be aware of these risk factors and use appropriate caution during treatment. If adverse bleeding occurs during infusion therapy, tenecteplase should be immediately terminated and blood products (fresh frozen plasma or cryoprecipitate) administered to reverse hypocoagulability.

Tenecteplase may also be used for treating thrombosed dialysis grafts. With this technique, the reconstituted tenecteplase is diluted with normal saline to the limits herein and injected into the graft. Systemic heparin (1,000-5,000 IU) is administered through a peripheral intravenous line or injected directly into the graft. Because of the observed prolonged pharmacologic activity of tenecteplase, prompt hemostasis at the puncture site may be difficult to achieve with use of only manual compression; therefore, placement of simple purse-string sutures at the catheter insertion site may be useful for providing immediate hemostasis. The sutures are removed in 24 hours by the dialysis nursing staff.

Studies with alteplase suggest that catheter-directed thrombolysis with tenecteplase can be safely and effectively used for treating DVT in patients having no contraindications to thrombolytic therapy. Because of substantial clot volume in DVT, higher doses of tenecteplase may be needed to effect lysis than those used for peripheral arterial occlusive disease (PAOD), e.g., a 1.0-5.0-mg bolus of tenecteplase infused directly into the clot followed by a continuous infusion of 0.003 mg/kg/h (weight-based regimen) or 0.25-0.5 mg/h (non-weight-based regimen).

Preferably, the solution herein is co-administered with another agent for treating the pathological collection, which agent depends on the type of disorder being treated. For example, in treating sepsis, an approved drug that could be combined with the tenecteplase is XIGRIS™ (drotrecogin alfa) (activated), a recombinant form of human Activated Protein C made by Eli Lilly & Co. While there is no approved therapy for the medical treatment of intracranial hemorrhage, evolving technology with synthetic cerebrospinal fluid (e.g., NEURYSOL™—Integra Lifes Sciences) mixed with alteplase has been shown to be effective in animals; alteplase has been delivered directly into the ventricles of the brain or in combination with endovascular coiling of aneurysms (Azmi-Ghadimi et al., Neurosurgery, 50: 421-4 (2002); Findlay et al., J. Neurosurg., 74: 803-807 (1991)).

Co-agents to treat peripheral thrombosis include a blood thinner, antiplatelet drug, or anti-coagulant drug or fibrinolytic agent such as heparin, warfarin, aspirin, tPA, tPA variants such as reteplase, streptokinase, urokinase, alfimeprase, factor Xa inhibitors, and GPIIb/IIIa platelet receptor antagonists such as, e.g., abciximab. Preferably, the agent is heparin, warfarin, aspirin, tPA, urokinase, reteplase, a factor Xa inhibitor, or a GPIIb/IIa platelet receptor antagonist, more preferably abciximab, eptifibatide, tirofiban hydrochloride, heparin, or warfarin.

Still more preferably the tenecteplase solution is administered at about 0.2 to 0.5 mg/hr with conservative use of heparin, for a maximum of 100 mg of tenecteplase. Low-dose intravenous heparin (about 300-600 units/hour, preferably about 500 units/hour) is preferably used to prevent pericatheter thrombosis.

The risk of adverse bleeding with the adjunctive use of heparin during tenecteplase infusion is unknown. The published literature shows widely varying doses of heparin—from none to therapeutic anticoagulation—with alteplase and no obvious trends have been found to predict adverse bleeding. The emerging clinical experience encompasses the use of subtherapeutic to full therapeutic doses of heparin. Overall, conservative use of heparin is recommended until further studies are performed and a subtherapeutic regimen is endorsed during tenecteplase therapy, e.g., 2500-IU heparin bolus followed by a continuous drip at 500 IU/h to maintain the partial thromboplastin time between 1.25 and 1.5 times control values. Because of the relatively prolonged duration of pharmacologic activity of tenecteplase, its infusion should be terminated at least one hour before removal of arterial sheaths if manual compression of the arterial access site is used. Systemic heparin therapy should begin at least four hours after sheath removal, if anticoagulation is desired and assuming there is no adverse bleeding at the catheter entry site.

Tenecteplase is incompatible and may precipitate when mixed directly with unfractionated heparin; concomitant heparin should be given through a separate intravenous line. An opaque diluent indicates precipitation of drug and may be associated with decreased efficacy.

The following detailed information is on the protein thrombolytics available for co-therapy with tenecteplase.

Streptokinase (SK, STREPTASE®; Aventis Behring LLC, King of Prussia, Pa.) is a purified derivative from Group C beta-hemolytic streptococci and indirectly activates plasminogen conversion (STREPTASE®. Full prescribing information. 2002 Physicians Desk Reference, Thomas Medical Economics Co., Montvale, N.J.). Streptokinase is approved for acute myocardial infarction, pulmonary embolism, systemic intravenous treatment of arterial and venous thrombosis, and dialysis cannula clearance. Despite its relatively low cost, SK has not been considered an optimal first-line lytic because of its antigenicity.

Urokinase (UK, Abbokinase®, Abbott Laboratories, Abbott Park, Ill.) is a thrombolytic derived from human kidney cells by tissue culture techniques, and is supplied as a lyophilized powder in 250,000-U vials and indicated for massive pulmonary embolism. UK was removed from the North American market in 1999 by the FDA due to manufacturing issues regarding source tissue procured from non-US sources. The US manufacturing facility has been substantially upgraded to meet Good Manufacturing Practice (GMP) standards mandated by the FDA, and the Agency approved its re-introduction in October 2002 with updated prescribing information. Despite measures to minimize risk of infection, urokinase may carry a risk of transmitting infectious agents, including those that cause the Creutzfeldt-Jakob disease or other diseases not yet known or identified; thus the risk of transmission of infectious agents cannot be totally eliminated. However, no case of transmission of infectious disease has ever been documented in the published literature of the estimated 4 million patients who have received urokinase.

For catheter-directed therapy, lyophilized UK (250,000-U vial) is reconstituted with 5 mL of sterile water and then diluted in normal saline (˜2000 U/mL). UK is infused at 60,000 U/hour (Mewissen et al., Radiology, 211: 39-49 (1999)); LeBlang et al., J. Vasc. Interv. Radiol., 3: 475-483 (1992); McNamara and Fischer, Am. J. Radiol., 144: 769-775 (1985)).

Alteplase (tPA, ACTIVASE®, CATHFLO™ ACTIVASE®, Genentech, Inc., South San Francisco, Calif.) is a tissue plasminogen activator (tPA) produced by recombinant DNA technology (ACTIVASE®, CATHFLO™ ACTIVASE®. Full prescribing information. 2002 Physicians Desk Reference, Thomas Medical Economics Co., Montvale, N.J.). It is a purified glycoprotein of 527 amino acids and is synthesized using complementary DNA (cDNA) for natural human tissue-type plasminogen activator obtained from an established human cell line. The manufacturing process involves secretion of the enzyme (alteplase) into the culture medium by an established mammalian cell line (Chinese hamster ovary cells) into which the cDNA has been genetically inserted. Alteplase is manufactured in 2-, 50-, and 100-mg vial configurations and is approved for use in catheter clearance, acute myocardial infarction, acute ischemic stroke, and pulmonary embolism.

For catheter-directed therapy, lyophilized alteplase is reconstituted in sterile water (1 mg/mL) and further diluted in normal saline (0.01-0.02 mg/mL) and infused at 0.5-1.0 mg/hr with conservative use of heparin (Semba et al., Tech. Vasc. Interv. Radiol., (2001), supra; Shortell et al. J. Vasc. Surg., 34: 854-859 (2001); Semba et al., J. Vasc. Interv. Radiol., pp. 279-287 (2000), supra).

Reteplase (RPA, RETAVASE®, Centocor, Inc., Malvern, Pa.) is a non-glycosylated deletion mutein of tissue plasminogen activator containing kringle 2 and the protease domains of human tPA (RETAVASE®. Full prescribing information. 2002 Physicians Desk Reference, Thomas Medical Economics Co., Montvale, N.J.). Reteplase contains 355 of 527 amino acids of native tPA and is produced by recombinant DNA technology in E. coli. The protein is isolated from inclusion bodies from E. coli, converted to its active form by an in vitro folding process, and purified by chromatographic separation. Reteplase is supplied as a lyophilized powder in a 10.8 U vial and is approved for acute myocardial infarction.

For catheter-directed therapy, lyophilized reteplase is reconstituted in sterile water (1 U/mL) and further diluted in normal saline (0.04 U/mL) and infused at 0.5-1.0 U/hr with conservative use of heparin (Castaneda et al., supra; Ouriel et al., J. Vasc. Interv. Radiol., 11: 849-854 (2000)).

Additionally, a tPA mutant designated BB-10153 (British Biotech) has been disclosed as useful for treatment of peripheral arterial disease (Dawson et al., J. Biol. Chem., 269: 15989-15992 (1994)) and may be useful in combination.

Such other drugs may be administered, by a route and in an amount commonly used therefor, contemporaneously or sequentially with the tenecteplase. When the tenecteplase is used contemporaneously with one or more other drugs, a pharmaceutical unit dosage form containing such other drugs in addition to the tenecteplase is preferred. Such additional molecules are suitably present or administered in combination in amounts that are effective for the purpose intended, typically less than what is used if they are administered alone without the tenecteplase.

The co-agent is administered to the mammal by any suitable technique including parenterally, by infusion, subcutaneously, intravenously, intranasally, orally, or by any other effective route. Preferably, the agent is given by infusion or orally.

Additional treatments include a combination of tenecteplase with mechanical devices or techniques, such as thrombolysis-facilitated rheolytic or high-speed maceration thrombectomy, mechanical thrombolysis, adjunctive percutaneous techniques such as rotational atherectomy and cutting balloon angioplasty, and stent placement. Recently, in vitro experiments have been performed assessing the viability of lytic protein solutions when propelled through rheolytic (ANGIOJET®, Possis Inc.) and macerating (CLOTBUSTER®, MicroVena, Inc.) catheters (Semba et al., J. Vasc. Interv. Radiol., 13 (2) (part 2): S76 (February 2002); Allie et al., 2002, supra). With the rheolytic device, alteplase remains fully active—however, significant protein degradation occurred through the macerating device.

Initial clinical investigation has suggested that thrombolysis-assisted rheolytic thrombectomy is a potential technique to accelerate clot lysis (Allie et al., 2002, supra). This would especially be pertinent in combination with thrombolytic therapy for limb salvage: mechanical thrombectomy (rheolytic thrombectomy—ANGIOJET™) and chemical thrombolysis (tenecteplase—TNKASE™) using the “power-pulse spray” technique. Percutaneous or open surgical techniques such as those necessitating an arteriotomy can be used to remove the thrombus in combination with the catheter-directed therapy, as well as pharmacomechanic methods, percutaneous aspiration thrombectomy (PAT), and percutaneous mechanical thrombectomy (PMT).

In addition, tenecteplase can be used with ultrasonic energy (e.g., low-power transverse cavitational ultrasound) to further accelerate thrombolysis.

The invention also provides kits. In one embodiment, the kits of the invention comprise a container of lyophilized tenecteplase, a container of sterile water for injection or bacteriostatic water for injection, and a container of normal saline, in predetermined amounts, in combination with a set of instructions, generally written instructions, relating to how to reconstitute and dilute the tenecteplase to the final concentration herein of about 0.01 to 0.05 m/mL. In a preferred embodiment, the amount of tenecteplase in the container is about 50 mg, the amount of water for injection is about 10 mL, and the instructions indicate that the tenecteplase is reconstituted to a final concentration of about 5 mg/mL.

Preferably, this kit will also contain instructions for exposing the diluted reconstituted tenecteplase to a catheter in an effective amount to treat a pathological collection of a fibrin-rich fluid. Preferably, the pathological collection is peripheral thrombosis sepsis, or adult respiratory distress syndrome. Such kit may optionally include a container comprising a co-agent for treating the pathological collection, such as heparin or warfarin or another co-agent as set forth above, with instructions for the combined use.

In a specific preferred embodiment, the kit further comprises instructions for delivering the diluted reconstituted tenecteplase in an effective amount to a mammal via a catheter to treat peripheral thrombosis. Such kit may optionally include a container containing, as active ingredient, a co-agent for treating peripheral thrombosis, as identified above, with instructions for its co-administration with the tenecteplase solution. A preferred such embodiment is a container comprising abciximab, eptifibatide, tirofiban hydrochloride, heparin, or warfarin with instructions for co-administration thereof in an effective amount with the diluted solution.

These further instructions included with the kit generally include information as to dosage, dosing schedule, and route of administration for the treatment of the thrombosis through delivery of the solution via a catheter. The containers of tenecteplase may be unit doses, bulk packages (e.g., multi-dose packages), or sub-unit doses.

In another embodiment, the kit comprises a container comprising a solution comprising tenecteplase, sterile water for injection or bacteriostatic water for injection, and normal saline, wherein the tenecteplase is present in a concentration of about 0.01 to 0.05 mg/mL, and instructions for exposing the solution in an effective amount to a pathological collection of a fibrin-rich fluid. As noted above, the kit may also comprise a container with a co-agent as active ingredient with instructions for the combined use thereof.

In this alternative kit, the instructions may be for delivering the solution in an effective amount to a mammal via a catheter to treat peripheral thrombosis. Such kit optionally includes a container comprising a co-agent for treating peripheral thrombosis, such as, for example, abciximab, eptifibatide, tirofiban hydrochloride, heparin, or warfarin, with instructions for co-administration thereof in an effective amount with the solution.

In these kits, tenecteplase may be packaged in any convenient, appropriate packaging. For example, if the tenecteplase is a freeze-dried formulation, an ampoule or vial with a resilient stopper is preferably used as the container, so that the drug may be easily reconstituted by injecting fluid through the resilient stopper. Ampoules with non-resilient, removable closures (e.g., sealed glass) or resilient stoppers are most conveniently used for solutions of tenecteplase ready for use in the catheter. In this latter case, the instructions preferably specify placing the contents of the vial in a catheter for immediate delivery.

Also contemplated are packages for use in combination with a specific device, such as those described by Allie et al., 2002, supra, with directions for such use.

Various features and aspects of the present invention are illustrated further in the examples that follow. While these examples are presented to show one skilled in the art how to operate within the scope of the invention, they are not intended in any way to serve as a limitation upon the scope of the invention. The disclosure of all citations herein is expressly incorporated herein by reference.

### EXAMPLE 1

Materials and Methods:

Commercial tenecteplase (50 mg/vial; TNK, TNKASE™, Genentech, Inc., South San Francisco, Calif.) was used for all studies. Assays were validated to standards and guidelines established by the United States Pharmacopeia (USP) and/or the United States Food and Drug Administration (FDA).

Freeze/Thaw Studies.

Frozen storage for 1 month at −20° C. Tenecteplase was reconstituted with 10 mL Sterile Water for Injection (SWFI), as defined by the United States Pharmacopeia (USP), to a final concentration of 5 mg/mL. The vial was swirled gently to dissolve the drug. Using a 10-mL syringe and 22-gauge needle, 2 mL (10 mg) of the reconstituted tenecteplase were dispensed into 5-mL glass vials (13 or 22 mm, Hollister-Stier Laboratories, Spokane, Wash.) in duplicates. The vials were stored at −20° C. in a non-cycling freezer. Samples were removed after 29 days of storage, thawed at ambient temperature (21°-22° C.) for approximately 4 hours, and assayed. Thawing times were recorded for vials set on the bench top alone at ambient temperature and by vials warmed by hand.

The quality of the assayed protein was compared to the Certificate of Analysis that characterizes the properties of commercially released lots of TNKASE™ submitted to the Food and Drug Administration, and compared to a freshly reconstituted commercial tenecteplase vial as control. Samples were assayed for optical clarity, pH, particle count, protein concentration, percent protein monomer, percent single-chain protein, and in-vitro clot lysis.

Frozen storage at −20° or −70° C. with 6 freeze/thaw cycles. Two sets of commercial tenecteplase vials were reconstituted with SWFI to a final concentration of 5 mg/mL. The solutions were gently mixed by swirling or inversion. The vials were set aside on the bench top under normal fluorescent light at ambient temperature (21°-22° C.) for 1-2 hours and then at 2°-8° C. for 2-4 hours before freezing one set at −70° C. and the other set at −20° C. for 24 to >100 hours. Afterwards, the vials were thawed at ambient temperature, stored at 2°-8° C., and frozen again as described before. The freezing and thawing cycle was repeated five times (six cycles total). Duplicate vials were performed for each cycle. At the end of cycles 1, 3, and 6, the vials were pulled and stored at −20° C. or −70° C. until analysis. The quality of the protein was compared to the Certificate of Analysis and to a freshly reconstituted commercial tenecteplase vial as control. Samples were assayed for optical clarity, pH, particle count, protein concentration, percent protein monomer, percent single-chain protein, and in-vitro clot lysis.

Dilution Studies.

Commercial tenecteplase (50 mg/vial) was reconstituted to 5 mg/mL with SWFI. Using a 3- or 5-mL syringe and 22-gauge needle, 1, 2, and 5 mL of normal saline (NS) were removed from separate 500-mL commercial normal-saline intravenous (IV) bags (Baxter, Inc., Deerfield, Ill.) in duplicates. The same volume was replaced with the reconstituted tenecteplase to give a final targeted concentration inside the bags of 0.01, 0.02, and 0.05 mg/mL. The bags were mixed gently by inversion. A 10-mL aliquot of the diluted tenecteplase solution was sampled via the IV port with a 10-mL syringe and dispensed into two 5-mL clean clear glass vials and assayed (T=0 hours). The IV bags containing the tenecteplase were placed on the bench top under normal fluorescent light at ambient temperature for 24 hours. Additional 10-mL aliquots were obtained from the bags after 8 and 24 hours for assay in duplicates. Samples were assayed for optical clarity, pH, protein concentration, and in-vitro clot lysis activity.

Assays.

Optical clarity. All samples in clean, clear glass vials were visually inspected for color and particulate matters. The inspection was performed against a black and white background under fluorescent light. The qualitative results were used to assess for visible precipitates.

pH. The pH determination was performed using a pH meter (Model PHM 93™, Radiometer Analytical, Inc. Lyon, France) and a microelectrode (Model MI-410™, Microelectrodes, Inc., Bedford, N.H.). The pH meter was standardized with pH 4.01 and 7.00 buffer standards prior to measurement at ambient room temperature.

Particle counts. Particulate matter analysis for small-volume parenterals was performed using a liquid particle counter (Model 9703 HIAC-ROYCO™; Pacific Scientific Instruments, Grants Pass, Oreg.). Samples were mixed in a clean, clear glass vial inside a laminar flow hood. Four measurements of the same sample were taken. The results are either “pass” or “fail” based upon standards established by the USP and the FDA.

Protein concentration. Analysis was performed using high-performance liquid chromatography (HPLC) (frozen storage for 1 month and dilution studies) or ultraviolet (UV) spectrophotometry (six cycle freeze/thaw studies). For all assays involving frozen samples, the sample aliquots were compared to tenecteplase reference material (which is the same as commercially available tenecteplase) and all values obtained were normalized to the tenecteplase reference material.

Concentration by HPLC: samples were diluted in singlet to assay range (0.5-10 μg) with standardized assay diluent. Tenecteplase reference material was diluted to 2.5 μg/mL as an internal reference. All values obtained were normalized to the tenecteplase reference material.

Concentration by UV spectrophotometry: protein concentration was determined by UV absorption at 280 nm. An ultraviolet/visible spectrum diode-array spectrophotometer (HP8254A™; Agilent Technologies, Palo Alto, Calif.) was employed with a 1-cm path length quartz cuvette for measurement. The measurement was blanked against the proper reference and a UV scan from 240-400 nm was obtained. The protein concentration was calculated as follows: Concentration (mg/mL)=(absorption at 280 nm−absorption at 320 nm)/1.9, where 1.9 is the absorptivity of tenecteplase at 280 nm (mL/mg-cm).

Protein monomer. Percent monomer was determined by native size-exclusion chromatography using a TSK Model 3000 SWXL™ column (Tosoh Biosep, Montgomeryville, Pa.; 300 mm×7.5 mm, 5 μm) employed on a HP1090™ liquid chromatogram (LC; Agilent Technologies) equipped with a diode-array detector. The mobile phase was 0.2 M arginine, 0.15 Tris buffer, 5% 1,2-propanediol, with pH 7.0±0.1. The flow rate was 0.5 mL/min. The samples (25-μg tenecteplase equivalent) were injected in singlet and the chromatograms collected with detection at 280 nm. The percent monomer was calculated as follows: % monomer=(monomer peak area/total peak area attributed to protein)×100. The assay was designed to determine the amount of protein aggregation and fragmentation of the protein.

Single-chain protein. Percent single-chain protein was determined by reduced size-exclusion chromatography using a TSK Model 3000 SWXL™ column (Tosoh Biosep; 300 mm×7.5 mm, 5 μm) employed on a HP1090 LC™ liquid chromatogram (Agilent Technologies) equipped with a diode-array detector. The mobile phase was 0.2 M sodium phosphate, 0.1% sodium dodecyl sulphate, pH 6.8. The flow rate was 1 mL/min. The samples (12.5-μg tenecteplase) were incubated with 20 mM dithiothreitol (final concentration) at 37° C. for 3-5 minutes and injected in singlet. The chromatograms were monitored with detection at 214 nm. The percent single chain (SC) was calculated as follows: % SC=(first main peak area/sum of the first and second main peak area)×100. The assay was designed to determine the amount of autolytic activity of the protease enzyme into single- and double-chain forms.

Bioactivity. In vitro bioactivity of tenecteplase was determined by a clot-lysis assay performed using a microplate reader (Model J21196 THERMOMAX™; Molecular Devices Co., Sunnyvale, Calif.). The samples were diluted to two different concentrations within the assay range (200-600 ng tenecteplase/mL) with standardized diluent. Tenecteplase reference material was used as an internal reference. All values obtained were normalized to the reference material and quantifiable as International Units (IU) per milligram.

Results:

Freeze/Thaw Studies.

Frozen storage for 1 month at −20° C. A summary of results is shown in Table 1. Thawed tenecteplase aliquots met Certificate of Analysis specifications and were similar to freshly reconstituted tenecteplase control samples. Time to thaw (defined as the time required until no visible ice formation is identified) was approximately 25 minutes on the bench top and 5 minutes when warmed by hand.

Frozen storage at −20° or −70° C. with 6 freeze/thaw cycles. A summary is shown in Table 2. Thawed tenecteplase aliquots at both storage temperatures met Certificate of Analysis specifications and were similar to freshly reconstituted tenecteplase control samples.

Dilution Studies.

The results are summarized in Table 3. The dilutions of tenecteplase were clear and colorless and without precipitates at all dilutions and times. Protein recovery was 70-75%, 80-85%, and 94-95% of the targeted concentration of 0.01, 0.02, and 0.05 mg/mL, respectively. The mean activity of the recovered protein at 24 hours, as determined by in vitro clot lysis assay, was 100%, 97%, and 92% for the 0.01, 0.02, and 0.05 mg/mL dilutions, respectively.

TABLE 1
Stability of aliquoted tenecteplase in glass vials stored for 1 month at −20° C.

| | | | Protein | Protein | Single-chain | Clot lysis × | Thawing |
|---|---|---|---|---|---|---|---|
| | Optical | | concentration | monomer | protein | 10² | Time |
| | clarity | pH | (mg/mL) | (%) | (%) | (IU/mg) | (min) |
| Control¹ | co/cl | 7.33 ± 0 | 5.12 ± 0.02 | 99.7 ± 0.05 | 65.2 ± 0 | 1.83 ± 0.01 | — |
| (N = 2) | | | | | | | |
| Aliquot^{1,2} | co/cl | 7.33 ± 0 | 5.05 ± 0.12 | 99.5 ± 0.17 | 64.9 ± 0.29 | 2.06 ± 0.06 | ˜25 |
| (N = 4) | | | | | | | (bench top) |
| | | | | | | | ˜5 |
| | | | | | | | (hand) |

¹Freshly reconstituted tenecteplase to 5 mg/mL with SWFI. Meets Certification of Analysis specifications for tenecteplase.
²2 mL aliquots of 5 mg/mL tenecteplase into 5 mL, 13-mm glass vials (N = 2) and 5 mL, 20-mm glass vials (N = 2).
Abbreviations: co, colorless; cl, clear

TABLE 2
Stability of aliquoted tenecteplase stored in glass vials following 6 freeze/thaw cycles at −20° C. or −70° C.

| | | | | | | Protein | Protein | Single chain | Clot lysis × |
|---|---|---|---|---|---|---|---|---|---|
| | | Optical | | Particle count | | concentration² | monomer | protein | 10² |
| | | Clarity | pH | ≧10 μm/vial | ≧25 μm/vial | (mg/mL) | (%) | (%) | (IU/mg) |
| Control¹ | | co/cl | 7.4 | 200 | 0 | 5.1 | 98 | 63 | 2.12 |
| −20° C. | Cycle 1 | co/cl | 7.3 | 1050 ± 72 | 10 ± 0 | 5.0 | 98 | 64 | 2.17 ± 0.05 |
| | Cycle 3 | co/cl | 7.3 | 200 ± 23 | 0 | 4.9 | 98 | 64 | 2.19 ± 0.07 |
| | Cycle 6 | co/cl | 7.3 | 155 ± 15 | 0 | 5.0 | 98 | 64 | 2.21 ± 0.01 |
| −70° C. | Cycle 1 | co/cl | 7.3 | 1080 ± 69 | 5 ± 5 | 5.1 | 98 | 64 | 2.20 ± 0.01 |
| | Cycle 3 | co/cl | 7.3 | 150 ± 30 | 5 ± 5 | 5.1 | 98 | 63 | 2.18 ± 0.05 |
| | Cycle 6 | co/cl | 7.3 | 185 ± 65 | 0 | 4.9 | 98 | 63 | 2.21 ± 0.05 |

¹Freshly reconstituted tenecteplase to 5 mg/mL with SWFI. Meets Certification of Analysis specifications for tenecteplase.
Abbreviations: co, colorless; cl = clear

TABLE 3
Stability and bioactivity of tenecteplase diluted in 500-mL normal
saline bags stored at ambient temperature for 24 hours.

| | | | | Protein | | | |
|---|---|---|---|---|---|---|---|
| | Targeted | | | Concentration¹ | | Clot Lysis Activity | |
| Time | Concentration | Optical | pH | μg/mL | % Mean Relative | μg/mL | % Mean Specific |
| (hrs) | (mg/mL) | Clarity | (mean) | (mean) | Concentration² | (mean) | Activity³ |
| 0 | 0.01 | co/cl | 6.72 | 7.0 | 70 | 6.0 | 86 |
| | 0.02 | co/cl | 6.88 | 17.0 | 85 | 15.0 | 88 |
| | 0.05 | co/cl | 7.14 | 47.5 | 95 | 40.5 | 85 |
| 8 | 0.01 | co/cl | — | 7.5 | 75 | 7.5 | 100 |
| | 0.02 | co/cl | — | 16.0 | 80 | 14.0 | 88 |
| | 0.05 | co/cl | — | 47.0 | 94 | 39.0 | 83 |
| 24 | 0.01 | co/cl | 6.74 | 7.0 | 70 | 7.0 | 100 |
| | 0.02 | co/cl | 6.88 | 16.0 | 80 | 15.5 | 97 |
| | 0.05 | co/cl | 7.09 | 47.0 | 94 | 43.0 | 92 |

¹Protein concentration as determined by high-performance liquid chromatography.
²Percent concentration relative to targeted concentration.
³Specific activity (μg/mL) = (clot lysis (mg/mL)/concentration (mg/mL)) × 100.
Abbreviations: co, colorless; cl, clear

Discussion:

Initial dose-ranging studies with tenecteplase as a thrombolytic appear to support the concept that tenecteplase can be empirically diluted in saline and maintain biological activity with acceptable rates of adverse events (Razavi et al., J. Vasc. Interv. Rad., supra). A protocol using a 0.01 mg/mL dilution in normal saline infused at 25 to 50 mL/hr (0.25-0.5 mg/hr) has been reported with angiographic efficacy in arterial and venous clot lysis (Razavi et al., J. Vasc. Interv. Rad., supra). Such doses of tenecteplase are safe and effective in peripheral catheter-directed thrombolytic therapy of arterial occlusions and deep vein thrombosis (Razavi et al., J. Endovasc. Ther., supra). The stability and bioactivity of these dilutions and confirmation that tenecteplase can be frozen and stored in small aliquots has been published (Semba et al., J. Vasc. Interv. Radiol., (2002), page S75, supra).

Additional successful clinical treatment studies using diluted tenecteplase solutions are disclosed by Allie et al., 2003, supra; Burkart et al., 2002, supra; and Burkart et al., 2003, supra, as discussed above.

In the studies herein, tenecteplase demonstrated retention of its baseline biological activity and biochemical properties when kept in frozen aliquots for up to one month, as well as when subjected to six freeze/thaw cycles. The number of freeze-thaw cycles chosen was to test the stability of the molecule well beyond the reasonable standards of clinical practice. The thawed solutions met all tenecteplase product specifications, and there was no evidence of increased proteolytic degradation or protein aggregation. Since tenecteplase has greater potency than alteplase, without being limited to any one theory, it is believed that a patient undergoing catheter-directed thrombolysis with tenecteplase would require significantly less than 50 mg (e.g., an infusion of 0.25 mg/hr over 15 hours is 3.75 mg). To prevent wastage of unused tenecteplase, small frozen aliquots can be performed similar to prior practices with alteplase (Calis et al., Am. J. Health Syst. Pharm., 56: 2056-2057 (1999)).

Dilution studies (up to 0.01 mg/mL) support that tenecteplase remains biochemically active up to 24 hours at ambient (room) temperature when diluted in commercial saline bags using techniques commonly employed in interventional practices. The protein concentrations in the bag were less than the targeted concentrations due in part to overfill of the saline bags (up to 10%) and protein adsorption that can occur on the polymer surface of the IV bag (Cornelius et al., J. Biomed. Mater. Res., 15: 622-632 (2000)). No visual precipitates were identified at any of the tested dilutions.

Tenecteplase at highly diluted doses delivered via catheter is expected to be successful in effecting peripheral arterial and venous thrombolysis, with few bleeding complications.

The most significant clinical risk associated with thrombolytic therapy is hemorrhage, which can substantially influence decision-making in the treatment of acute arterial or venous occlusions (Ricotta et al., J. Vasc. Surg., 6: 45-50 (1987)). Because tenecteplase has higher fibrin specificity than other agents, it is expected that use thereof will provide a low major hemorrhagic complication rate comparing favorably with reports of other agents. In a survey of the clinical trials using tPA for peripheral thrombolysis, Semba et al., J. Vasc. Interv. Radiol., (2000), pp 149-161, supra, estimated the incidence of major adverse bleeding to be 5.1% (range 0 to 17%). A similar rate of major hemorrhage (5.6%) was reported in the Surgery versus Thrombolysis for Ischemia of the Lower Extremity (STILE) trial, where both urokinase and tPA were used (STILE trial, Ann. Surg., supra). In the National Audit of Thrombolysis for Acute Limb Ischemia (NATALI) registry from Great Britain and Ireland, an 11.9% major complication rate was observed (Thomas et al., Br. J. Surg., 86: 710 (1999)). The Thrombolysis or Peripheral Arterial Surgery (TOPAS) Trial, which compared recombinant urokinase with surgery, reported a major bleeding rate of 12.5%, with 1.6% intracranial hemorrhage (ICH) in the thrombolytic group (Ouriel et al., N. Engl. J. Med., 16: 1105-1111 (1998)).

Other single-institution reports have presented relatively high minor and major hemorrhagic complications during peripheral thrombolysis. In one analysis of 653 patients, transfusion was necessary in 15% of patients during the course of thrombolysis (Ouriel et al., J. Vasc. Interv. Radiol., 11: 295-298 (2000), supra). Access site hematoma requiring surgical intervention was reported in 4.9% of the patients. In the report by Suggs et al., Am. J. Surg., 178: 103-106 (1999)), 4 (7%) of their 57 patients treated with urokinase had hemorrhagic complications requiring blood transfusions. Two additional patients developed groin complications necessitating additional procedures.

Although the data regarding the use of therapeutic levels of heparin is inconclusive, it has been suggested that it is associated with an increased rate of hemorrhagic complications (Ouriel et al., supra (1998); Decrinis et al., Eur. Heart J., 14: 297-305 (1993)), so use of less heparin is advised if possible.

Alternative to heparin, a trial of a GPIIb/IIIa platelet receptor antagonist or inhibitor in conjunction with tenecteplase may prove successful with the highly diluted solution of tenecteplase herein. In fact, a pilot study of reteplase in combination with abciximab found 93% technical and clinical success with no major hemorrhagic complications (Drescher et al., J. Vasc. Interv. Radiol., 13: 37-43 (2002)), and a pilot study of tenecteplase and eptifibatide showed clinical success of the combination for peripheral arterial and venous thrombolysis (Burkart et al., 2003, supra).

The clot lysis percent in PAO patients for tenecteplase is expected to be comparable to that of reteplase (Davidian et al., supra; Ouriel et al., supra (2000), pp. 849-854), urokinase (Ouriel et al., supra (1998); Suggs et al., supra), and tPA (Thomas et al., supra; Arepally et al., supra; Semba et al., supra, J. Vasc. Interv. Radiol., pp. 149-161 (2000)). Tenecteplase may be a suitable agent when employing a bolus injection or relying entirely on an initial large intraclot dose, as advocated by Chang et al., J. Vasc. Interv. Radiol., 12: 247-252 (2001).

In conclusion, the data herein support the practice of freezing and aliquotting tenecteplase and the use of diluted solutions as a thrombolytic agent for such applications as peripheral arterial or venous thrombosis, flushing occluded catheters, etc. Semba et al., J. Vasc. Interv. Radiol., 14: 475-479 (2003) discloses the studies shown herein by example.

What is claimed is:

1. A solution comprising about 0.01 to 0.05 mg/mL of tenecteplase in sterile water for injection or bacteriostatic water for injection and normal saline.

2. The solution of claim 1 wherein the tenecteplase is in a concentration of about 0.01 to 0.04 mg/mL.

3. The solution of claim 1 wherein the tenecteplase is in a concentration of about 0.01 to 0.03 mg/mL.

4. The solution of claim 1 wherein the tenecteplase is in a concentration of about 0.01 to 0.02 mg/mL.

5. The solution of claim 1 wherein the tenecteplase is in a concentration of about 0.01 to 0.015 mg/mL.

6. The solution of claim 1 wherein the tenecteplase is in sterile water for injection.

7. A catheter comprising the solution of claim 1.

8. A method for treating a pathological collection of a fibrin-rich fluid comprising exposing the fluid to an effective amount of a solution comprising about 0.01 to 0.05 mg/mL of tenecteplase in sterile water for injection or bacteriostatic water for injection and normal saline.

9. The method of claim 8 wherein the tenecteplase is in sterile water for injection.

10. The method of claim 8 wherein the fluid is exposed in vivo or ex vivo.

11. The method of claim 8 wherein the pathological collection is contained within a catheter.

12. The method of claim 11 wherein the catheter is flushed with the solution.

13. The method of claim 12 wherein the catheter is contacted with the solution for at least about five days to remove fibrin-bound blood clots.

14. The method of claim 8 wherein the fluid is exposed in vivo by administration to a mammal.

15. The method of claim 14 wherein the mammal is a human.

16. The method of claim 14 further comprising administering to the mammal an effective amount of a co-agent for treating the pathological collection.

17. The method of claim 14 wherein the pathological collection being treated is sepsis or acute respiratory distress.

18. The method of claim 14 wherein the pathological collection is contained within a catheter.

19. The method of claim 18 wherein the pathological collection being treated is peripheral thrombosis and the catheter is indwelling.

20. A method for treating peripheral thrombosis in a mammal comprising delivering to the mammal via a catheter an effective amount of a solution comprising about 0.01 to 0.05 mg/mL of tenecteplase in sterile water for injection or bacteriostatic water for injection and normal saline.

21. The method of claim 20 wherein the catheter is placed in a blood clot in the mammal.

22. The method of claim 20 further comprising administering to the mammal an effective amount of a co-agent for treating the thrombosis.

23. The method of claim 22 wherein the co-agent is a blood thinner, anti-platelet drug, or anti-coagulant drug.

24. The method of claim 23 wherein the co-agent is heparin, warfarin, aspirin, tissue-plasminogen activator, urokinase, reteplase, or a glycoprotein (GP) IIb/IIIa platelet receptor antagonist.

25. The method of claim 24 wherein the co-agent is abciximab, eptifibatide, tirofiban hydrochloride, heparin, or warfarin.

26. The method of claim 25 wherein the co-agent is administered via infusion or orally.

27. A kit comprising a container comprising lyophilized tenecteplase, a container comprising sterile water for injection or bacteriostatic water for injection, a container comprising normal saline, and instructions for reconstituting the tenecteplase with the water for injection and diluting the reconstituted tenecteplase with the normal saline to a final concentration of about 0.01 to 0.05 mg/mL of tenecteplase.

28. The kit of claim 27 wherein the container with tenecteplase contains about 10-50 mg tenecteplase, the container with water for injection contains about 2-10 mL of such water, and the instructions indicate that the tenecteplase is reconstituted to a final concentration of about 5 mg/mL.

29. The kit of claim 27 further comprising instructions for exposing the diluted reconstituted tenecteplase to a catheter in an effective amount to treat a pathological collection of a fibrin-rich fluid.

30. The kit of claim 29 wherein the pathological collection is peripheral thrombosis, sepsis, or adult respiratory distress syndrome.

31. The kit of claim 27 further comprising instructions for delivering the diluted reconstituted tenecteplase in an effective amount to a mammal via a catheter to treat peripheral thrombosis.

32. The kit of claim 31 further comprising a container comprising abciximab, eptifibatide, tirofiban hydrochloride, heparin, or warfarin with instructions for co-administration thereof in an effective amount with the diluted tenecteplase.

33. A kit comprising a container comprising a solution comprising about 0.01 to 0.05 mg/mL of tenecteplase in sterile water for injection or bacteriostatic water for injection and normal saline, and instructions for exposing the solution in an effective amount to a pathological collection of a fibrin-rich fluid.

34. The kit of claim 33 wherein the instructions are for delivering the solution in an effective amount to a mammal via a catheter to treat peripheral thrombosis.

35. The kit of claim 34 further comprising a container comprising abciximab, eptifibatide, tirofiban hydrochloride, heparin, or warfarin with instructions for co-administration thereof in an effective amount with the solution.
